(19) United States
(12) Patent Application Publication
(10) Pub. No.: US 2010/0312110 A1
(43) Pub. Date: Dec. 9, 2010

(54) ULTRASONOGRAPH

(75) Inventors: Takao Suzuki, Kanagawa (JP); Hisashi Hagiwara, Kanagawa (JP); Makoto Kato, Kanagawa (JP); Yoshinao Tan-naka, Kanagawa (JP)

Correspondence Address: MARK D. SARALINO (PAN);RENNER, OTTO, BOISSELLE & SKLAR, LLP, 1621 EUCLID AVENUE, 19TH FLOOR, CLEVELAND, OH, 44115, US

(73) Assignee: Panasonic Corporation

(21) Appl. No.: 12/160,447

(22) PCT Filed: Jan. 10, 2007
(86) PCT No.: PCT/JP2007/050126

§ 371 Date: Jul. 22, 2010

(30) Foreign Application Priority Data
Jan. 11, 2006 (JP) 2006-003913

(51) Int. Cl. A61B 8/14

(52) U.S. Cl. 600/443

(57) ABSTRACT

An ultrasonic diagnostic apparatus according to the present invention includes: a transmitting section 102 driving an ultrasonic probe; a receiving section 103 receiving an ultrasonic echo, produced by getting an ultrasonic wave reflected by a subject, at the probe to generate a received signal; a tissue tracking section 105 for tracking the motion of each tissue of the subject based on the received signal and outputting location tracking information; a radius and wall thickness calculating section calculating the radius and wall thickness of the blood vessel; a tissue attribute value calculating section 108 calculating radial and circumferential elasticities of the vascular wall of the vessel based on information about the blood pressure of the subject provided externally, the tracking information, and the radius and wall thickness of the vessel; and a display section 107 presenting the radial and circumferential elasticities. While the transmitting section and the receiving section are transmitting or receiving the ultrasonic wave, the tissue attribute value calculating section calculates the radial elasticities one after another based on the tracking information and the information about the blood pressure, and the display section presents the radial elasticities. But while the transmission and reception of the ultrasonic waves are suspended, the tissue attribute value calculating section calculates the circumferential elasticity based on the tracking information, the information about the blood pressure, and the radius and wall thickness of the blood vessel and the display section presents the circumferential elasticities.

### TECHNICAL FIELD

The present invention generally relates to an ultrasonic diagnostic apparatus, and more particularly relates to an ultrasonic diagnostic apparatus for calculating the elasticity of a subject's tissue based on a tracking waveform obtained by tracking the motion of the tissue.

### BACKGROUND ART

An ultrasonic diagnostic apparatus is used to make a noninvasive checkup on a subject by irradiating him or her with an ultrasonic wave and analyzing the information contained in its echo signal. For example, a conventional ultrasonic diagnostic apparatus that has been used extensively converts the intensity of an echo signal into its associated pixel luminance, thereby presenting the subject's structure as a tomographic image. In this manner, the internal structure of the subject can be known.

Meanwhile, some people are attempting recently to track the motion of a subject's tissue more precisely and evaluate the strain and the elasticity, viscosity or any other physical (attribute) property of the tissue mainly by analyzing the phase of the echo signal.

Patent Document No. 1 discloses a method for tracking a subject's tissue highly precisely by calculating the magnitude of instantaneous displacement of a local region of the subject based on the phase difference of an ultrasonic echo signal to be transmitted and received at regular intervals and by summing the magnitudes of displacements together. Hereinafter, a method for tracking a subject's tissue as disclosed in Patent Document No. 1 will be described with reference to FIG. 3. Suppose ultrasonic pulses are transmitted toward the same location of a subject at regular intervals ΔT and received signals, generated by converting the resultant echo signals into electrical signals, are identified by y(t) and y(t+ΔT), respectively, where t represents the receiving time when the transmitting time is zero. The echo signal obtained from a measuring point, which is located at a distance (or depth) x from the probe, and its receiving time tx satisfy the following Equation (1), in the case where C is the sonic velocity.

tx=x/(C/2) (1)

In this case, supposing the phase difference between y(tx) and y(tx+ΔT) is Δθ and the center frequency of the ultrasonic wave around tx is f, the magnitude of displacement Δx of the measuring point during this interval ΔT is represented by the following Equation (2):

Δx=−C·Δθ/4πf (2)

The location x′ of the measuring point after the interval ΔT has passed is given by adding the magnitude of displacement Δx, given by Equation (2), to the original measuring point x as in the following Equation (3):

x′=x+Δx (3)

By performing this calculation repeatedly, the location of the measuring point in the subject can be tracked. For example, if the receiving time of the echo reflected from the location x′ is tx′ and the received signal that has been transmitted and received next is y(t+2ΔT), the location x″ of the measuring point in 2ΔT can be calculated based on the phase difference between y(tx′+ΔT) and y(tx′+2ΔT) by Equations (1) and (2).

Patent Document No. 2 further develops the method of Patent Document No. 1 into a method of calculating the elasticity of a subject's tissue (e.g., an arterial vascular wall, in particular). According to this method, first, an ultrasonic wave is transmitted from a probe 101 toward the blood vessel 222 of a subject 230 as shown in FIG. 4(a). And the echo signals, reflected from measuring points A and B on the vascular wall of the blood vessel 222, are analyzed by the method of Patent Document No. 1, thereby tracking the motions of the measuring points A and B. FIG. 4(b) shows the tracking waveforms TA and TB of the measuring points A and B along with an electrocardiographic complex ECG.

As shown in FIG. 4(b), the tracking waveforms TA and TB have the same periodicity as the electrocardiographic complex ECG, which shows that the artery dilates and contracts in sync with the cardiac cycle of the heart. More specifically, when the electrocardiographic complex ECG has outstanding peaks called “R waves”, the heart starts to contract, thus pouring vascular flow into the artery and raising the blood pressure. As a result, the vascular wall is dilated rapidly. That is why soon after the R wave has appeared on the electrocardiographic complex ECG, the artery dilates rapidly and the tracking waveforms TA and TB rise steeply, too. After that, however, as the heart dilates slowly, the artery contracts gently and the tracking waveforms TA and TB gradually fall to their original levels. The artery repeats such a motion cyclically.

The difference between the tracking waveforms TA and TB is represented as a waveform W showing a variation in thickness between the measuring points A and B. Supposing the maximum variation of the thickness variation waveform is ΔWand the reference thickness between the measuring points A and B during initialization (i.e., the end of the diastole) is Ws, the magnitude of maximum strain ε between the measuring points A and B is calculated by the following Equation (4):

ε=ΔW/Ws (4)

As this strain is caused due to the difference between the blood pressures applied to the vascular wall, the elasticity Er between the measuring points A and B is given by the following equation (5), assuming that ΔP is the blood pressure difference at this time.

Er=ΔP/ε=ΔP·Ws/ΔW (5)

Therefore, by measuring the elasticity Er for multiple spots on a tomographic image, an image representing the distribution of elasticities can be obtained. If an atheroma 220 has been created in the vascular wall of the blood vessel 222 as shown in FIG. 4(a), the atheroma 220 and its surrounding vascular wall tissue have different elasticities. That is why if an image representing the distribution of elasticities is obtained, important information can be obtained in inspecting the attribute of the atheroma (e.g., how easily the atheroma may rupture, among other things).

The elasticity thus obtained is called the “radial elasticity of blood vessel”. A cylindrical vascular wall has three types of elasticities—not only the radial elasticity Er but also a circumferential elasticity Eθ and axial elasticity Ez. The elasticity Er obtained by the method of Patent Document No. 2 is the radial elasticity of a vascular wall. In the radial direction of a vascular wall in which pressure is applied, the strain of the vascular wall is detected and the elasticity is figured out based on that strain and the elasticity.

Non-Patent Document No. 1 discloses a method for calculating the circumferential elasticity Eθ of a vascular wall. According to Non-Patent Document No. 1, the vascular wall has a concentric three-layer structure, and therefore, not so much the radial elasticity Er as the circumferential elasticity Eθ reflects the tissue attribute of the vascular wall more accurately. In Non-Patent Document No. 1, the circumferential elasticity Eθ is calculated by the following Equation (6), assuming that h0 is the initial radial thickness of the vascular wall and that r0 is the initial radius of the blood vessel.

Eθ=−(1/2)(r0/h0+1)(ΔP/ε) (6)
Patent Document No. 1: Japanese Patent Application Laid-Open Publication No. 10-5226
Patent Document No. 2: Japanese Patent Application Laid-Open Publication No. 2000-229078
Patent Document No. 3: Pamphlet of PCT International Application Publication No. 2004/110280
Non-Patent Document No. 1: Hideyuki Hasegawa et al., “Evaluation of Regional Elastic Modulus of Cylindrical Shell with Nonuniform Wall Thickness”, J Med Ultrasonics, Vol. 28, No. 1 (2001), pp. J3 to J13
Non-Patent Document No. 2: Hiroshi Kanai, edited by the Acoustical Society of Japan, “Spectral Analysis of Sounds and Vibrations”, Corona Publishing Co., Ltd., ISBN4-339-01105-3
Non-Patent Document No. 3: S. Timoshenko, “The Theory of Elasticity”, McGraw-Hill, 1970

### DISCLOSURE OF INVENTION

### Problems to be Solved by the Invention

As can be seen from Equation (6), to calculate the circumferential elasticity Eθ, the radius R0 and wall thickness h0 of the blood vessel need to be measured. In measuring the vascular wall with ultrasonic waves, however, the boundary between the vascular posterior wall (i.e., the vascular wall that is more distant from the probe) and the vascular flow can be detected clearly. But it is difficult to accurately detect the boundary between the vascular anterior wall (i.e., the vascular wall that is closer to the probe) and the vascular flow due to the influences of multiple reflection or ringing of ultrasonic waves. Consequently, the circumferential elasticity Eθ could not calculated exactly.

In order to overcome these problems of the prior art, the present invention has an object of providing an ultrasonic diagnostic apparatus that can calculate the circumferential elasticity precisely without using any expensive arithmetic logic unit and without giving the operator too much trouble.

### Means for Solving the Problems

An ultrasonic diagnostic apparatus according to the present invention includes: a transmitting section that drives an ultrasonic probe to transmit an ultrasonic wave toward a subject including a blood vessel; a receiving section that receives an ultrasonic echo, produced by getting the ultrasonic wave reflected by the subject, at the ultrasonic probe to generate a received signal; a tissue tracking section for tracking the motion of each tissue of the subject based on the received signal and outputting location tracking information; a radius and wall thickness calculating section that calculates the radius and wall thickness of the blood vessel; a tissue attribute value calculating section that calculates first and second types of tissue attribute values of the vascular wall of the blood vessel based on information about the blood pressure of the subject that has been provided externally, the location tracking information, and the radius and wall thickness of the blood vessel; and a display section that presents the first and second types of tissue attribute values. While the transmitting section and the receiving section are transmitting or receiving the ultrasonic wave, the tissue attribute value calculating section calculates the first type of tissue attribute values one after another based on the location tracking information and the information about the blood pressure, and the display section presents the first type of tissue attribute values. But while the transmission and reception of the ultrasonic waves are suspended, the tissue attribute value calculating section calculates the second type of tissue attribute value based on the location tracking information, the information about the blood pressure, and the radius and wall thickness of the blood vessel. In accordance with an instruction given by an operator, the tissue attribute value calculating section outputs the first and/or second type(s) of tissue attribute values, and the display section presents the first and/or second type(s) of tissue attribute values supplied from the tissue attribute value calculating section.

In one preferred embodiment, the second type of tissue attribute values needs more computations than the first type of tissue attribute values.

In this particular preferred embodiment, the first and second types of tissue attribute values are radial elasticity and circumferential elasticity, respectively. While the transmitting section and the receiving section are transmitting or receiving the ultrasonic waves, the tissue attribute value calculating section calculates the radial elasticities one after another based on the location tracking information and the information about the blood pressure. But while the transmission and reception of the ultrasonic waves are suspended, the tissue attribute value calculating section calculates the circumferential elasticities based on the location tracking information, the information about the blood pressure, and the radius and wall thickness of the blood vessel.

In a specific preferred embodiment, the radius and wall thickness calculating section locates a boundary between the vascular wall and vascular flow and a boundary between the vascular wall and a surrounding tissue based on at least one of the received signal and the location tracking information and calculates the radius and wall thickness of the blood vessel with respect to the boundary located.

In a more specific preferred embodiment, the ultrasonic diagnostic apparatus further includes: a tomographic image processing section that generates, based on the received signal, a tomographic image of the subject to present on the display section; and a user interface for entering information about the vascular wall-flow boundary and the vascular wall-surrounding tissue boundary into the radius and wall thickness calculating section by getting one of these boundaries specified by the operator on the tomographic image on the display section.

In another preferred embodiment, the ultrasonic diagnostic apparatus further includes: a tomographic image processing section that generates, based on the received signal, a tomographic image of the subject to present on the display section; and a user interface that allows the operator to enter either corrected ones of the vascular wall-flow boundary and vascular wall-surrounding tissue boundary that have been located by the radius and wall thickness calculating section by getting one of these two boundaries specified by the operator on the tomographic image on the display section or corrected ones of the radius and wall thickness of the blood vessel that have been calculated by the radius and wall thickness calculating section.

In this particular preferred embodiment, while the ultrasonic waves are not being transmitted or received, the display section continues to present the first type of tissue attribute value until the operator enters information into the radius and wall thickness calculating section through the user interface.

In a specific preferred embodiment, the ultrasonic diagnostic apparatus further includes a memory that stores at least one of the received signal, the location tracking information and the magnitude of strain calculated based on the location tracking information. While the ultrasonic wave is not being transmitted or received, the tissue tracking section and the tissue attribute value calculating section read at least one of the received signal, the location tracking information and the magnitude of strain from the memory, calculate the second type of tissue attribute value, and present the value on the display section.

In a more specific preferred embodiment, the tissue tracking section and the tissue attribute value calculating section read at least one of the received signal, the location tracking information and the magnitude of strain calculated based on the location tracking information at an arbitrary point in time from the memory, calculate the second type of tissue attribute value, and present the value on the display section.

In one preferred embodiment, the memory stores the second type of tissue attribute value calculated in association with the time.

In this particular preferred embodiment, if the second type of tissue attribute value is stored in the memory, the tissue attribute value calculating section reads the second type of tissue attribute value from the memory without receiving the operator's input through the user interface and the display section presents the second type of tissue attribute value.

### EFFECTS OF THE INVENTION

According to the present invention, the circumferential elasticity of the vascular wall can be calculated precisely by a simple procedure without using any expensive arithmetic-logic unit.

### BRIEF DESCRIPTION OF DRAWINGS

FIG. 1 is a block diagram illustrating a preferred embodiment of an ultrasonic diagnostic apparatus according to the present invention.

FIG. 2 illustrates an exemplary picture presented on the apparatus shown in FIG. 1.

FIG. 3 shows the principle of tracking the location of a tissue based on a phase difference.

FIG. 4(a) illustrates a procedure in which a vascular wall is measured with an ultrasonic diagnostic apparatus and

FIG. 4(b) illustrates exemplary tracking waveform and thickness variation waveform obtained by measuring.

### DESCRIPTION OF REFERENCE NUMERALS

100 control section
101 probe
102 transmitting section
103 receiving section
104 tomographic image processing section
105 tissue tracking section
106 image synthesizing section
107 monitor
108 tissue attribute value calculating section
109 memory
110 radius and wall thickness calculating section
111 blood pressure value getting section
122 user interface
200 tomographic image
201 elasticity image
202 tomographic reflection intensity scale
203 elasticity image scale
204 biomedical signal waveform

### BEST MODE FOR CARRYING OUT THE INVENTION

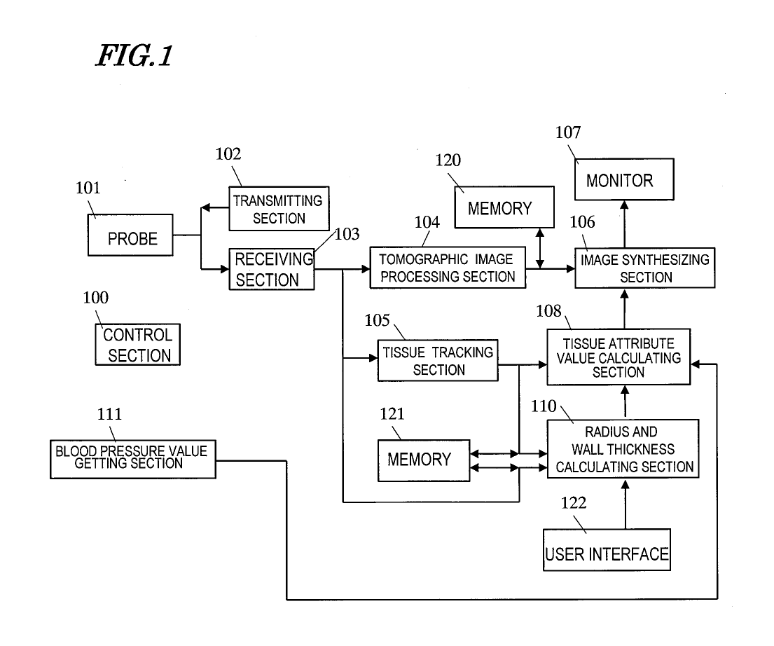

Hereinafter, a preferred embodiment of an ultrasonic diagnostic apparatus according to the present invention will be described. FIG. 1 is a block diagram illustrating an ultrasonic diagnostic apparatus according to the present invention. This ultrasonic diagnostic apparatus includes a transmitting section 102, a receiving section 103, a tomographic image processing section 104, a tissue tracking section 105, an image synthesizing section 106, a monitor 107, a tissue attribute value calculating section 108, a radius and wall thickness calculating section 110, memories 120 and 121 and a user interface 122. The ultrasonic diagnostic apparatus further includes a control section 100 that controls all of these components at predetermined timings and in a predetermined order. Although not shown, the control section 100 includes a user interface such as a keyboard, a trackball, a switch or a button.

Not all of these components shown in FIG. 1 have to be hardware circuits. For example, the functions of the control section 100, the tomographic image processing section 104, the tissue tracking section 105, the image synthesizing section 106, the tissue attribute value calculating section 108 and the radius and wall thickness calculating section 110 may be realized by a combination of a CPU and a software program.

A probe 101 that sends out an ultrasonic wave toward a subject and receives the reflected ultrasonic echo from the subject is connected to the transmitting section 102 and the receiving section 103. The ultrasonic diagnostic apparatus may either have a dedicated probe 101 or use a general probe as the probe 101. In the probe 101, arranged are a number of piezoelectric transducers. By changing the piezoelectric transducers to use and by adjusting the timing to apply a voltage to the piezoelectric transducer, the angle of deviation and focus of the ultrasonic wave to transmit and receive are controlled.

In accordance with the instruction given by the control section 100, the transmitting section 102 generates a high-voltage signal that drives the probe 101 at a specified timing. The probe 101 converts the signal that has been generated by the transmitting section 102 into an ultrasonic wave and sends out the ultrasonic wave toward a subject.

The ultrasonic echo that has been reflected by an internal organ of the subject is converted by the probe 101 into an electrical signal, which is then amplified by the receiving section 103 to generate a received signal. By changing the piezoelectric transducers to use in the probe 103 as described above, the receiving section 103 can detect only an ultrasonic wave that has come from a particular position (i.e., a particular focus position) or a particular direction (i.e., at a particular angle of deviation).

The tomographic image processing section 104 includes a filter, a detector, and a logarithmic amplifier, and analyzes mainly the amplitude of the received signal, thereby presenting the internal structure of the subject as an image. The tomographic image thus obtained is synthesized with an elasticity image by the image synthesizing section 106 and presented on the monitor 107 as a display section as will be described later.

The tissue tracking section 105 includes a memory to store the received signals, a magnitude of displacement calculating section for calculating, based on the phase difference between the received signals, the magnitude of displacement of the subject's tissue in the ultrasonic wave transmitting and receiving directions by Equation (1), and a tracking location calculating section that calculates the new location by adding the magnitude of displacement to the original location. The tissue tracking section 105 outputs the location tracking information of each tissue in the subject in the ultrasonic wave transmitting and receiving directions.

The radius and wall thickness calculating section 110 calculates the radius and wall thickness of the blood vessel of the subject. Specifically, by analyzing at least one of the location tracking information supplied from either the tissue tracking section 105 or the memory 121 and the received signal, the radius and wall thickness calculating section 110 detects the boundary of the tissue. Also, the radius and wall thickness calculating section 110 figures out the radius and wall thickness of the blood vessel by locating the boundary between the vascular wall and the surrounding tissue or the boundary between the vascular wall and the vascular flow. If the location tracking information is used, the outside diameter of the blood vessel can be obtained by calculating the difference in the amount of vascular flow, the thickness of the vascular wall and the tracking waveform from the surrounding tissue.

On the other hand, if the received signal is used, then the amplitude of the received signal is analyzed. The amplitude of the received signal represents the intensity of the wave reflected from a measuring region of the subject. That is why the intensities of the reflected waves are different between the vascular wall and the vascular flow or between the vascular wall and the surrounding tissue. For that reason, by detecting the difference in the amplitude of the received signal, the boundary can be detected and located.

The user interface 122 is an input section that allows the operator to correct the radius and wall thickness of the blood vessel that have been calculated by the radius and wall thickness calculating section 110. Specifically, the user interface is an input device such as a keyboard, a trackball or a mouse. By correcting, on the tomographic image presented on the monitor 107, the boundary between the vascular wall and the vascular flow or the boundary between the vascular wall and the surrounding tissue, which has been determined by the radius and wall thickness calculating section 110, with the user interface 122, the operator enters the corrected boundary between the vascular wall and the surrounding tissue or the corrected boundary between the vascular wall and the vascular flow into the radius and wall thickness calculating section. Optionally, not only the tomographic image and the boundary between the vascular wall and the vascular flow or between the vascular wall and the surrounding tissue that has been calculated by the radius and wall thickness calculating section 110 but also the radius and wall thickness of the blood vessel may be presented on the monitor 107 so as to allow the operator to enter appropriate radius and wall thickness values for the blood vessel through the user interface 122 while watching the screen of the monitor 107.

Also, if the radius and wall thickness calculating section 110 has failed to calculate the radius or wall thickness of the blood vessel with minimum required precision based on the location tracking information or the received signal, then the operator himself or herself may determine the boundary between the vascular wall and the vascular flow or the boundary between the vascular wall and the surrounding tissue on the tomographic image presented on the monitor 107 with the user interface 122. And in accordance with the boundary that has been determined by the operator, the radius and wall thickness calculating section 110 may calculate the radius and wall thickness of the blood vessel. In accordance with the input through the user interface 122, the radius and wall thickness calculating section 110 outputs the corrected radius and corrected wall thickness of the blood vessel to the tissue attribute value calculating section 108. Optionally, the user interface 122 may be a keyboard, a trackball, a switch or a button provided for the control section 100.

The tissue attribute value calculating section 108 receives the location tracking information from the tissue tracking section 105 and calculates the magnitude of strain by Equations (3) and (4). Also, the tissue attribute value calculating section 108 receives a blood pressure value from the blood pressure value getting section 111 and calculates the radial elasticity as a first type of tissue attribute value by Equation (5). The blood pressure value getting section 111 outputs the blood pressure value to the tissue attribute value calculating section 108. The blood pressure value getting section 111 may be either a blood pressure manometer to measure the blood pressure of the subject or an input device such as a keyboard that allows the operator to enter a blood pressure value.

The tissue attribute value calculating section 108 further receives the corrected radius and wall thickness values of the blood vessel from the radius and wall thickness calculating section 110 and calculates the circumferential elasticity as a second type of tissue attribute value by Equations (4) and (6). The radial and circumferential elasticities are calculated for each tissue of the subject. That is to say, the radial and circumferential elasticities are arranged as a two-dimensional map in the ultrasonic wave transmitting and receiving direction and in the direction perpendicular to the transmitting and receiving direction.

The circumferential elasticity cannot be obtained unless accurate radius and wall thickness of the blood vessel are entered. For that reason, as will be described in detail later, the circumferential elasticity is not calculated until the operator has corrected the radius and wall thickness of the blood vessel with the transmission and reception of the ultrasonic wave for measuring purposes stopped. Meanwhile, the radial elasticities are calculated one after another in real time with the ultrasonic wave transmitted and received for measuring purposes.

In this manner, the tissue attribute value calculating section 108 calculates the first type of tissue attribute value while the ultrasonic wave is being transmitted or received and calculates the second type of tissue attribute value while no ultrasonic waves are being transmitted or received. The first type of tissue attribute value could be either a property value that can be calculated relatively easily (i.e., that does not require too much computations) even while the ultrasonic wave is being transmitted or received or a property value that can be calculated automatically based on the received signal. On the other hand, the second type of tissue attribute value should be either a tissue attribute value that requires much more computation than the first type of tissue attribute value or a tissue attribute value that needs to be calculated in accordance with the corrections made by the operator. The tissue attribute value calculating section 108 calculates the second type of tissue attribute value while no ultrasonic waves are being transmitted or received. That is why compared to calculating the second type of tissue attribute value with ultrasonic waves transmitted or received, the load on the arithmetic logic unit of the ultrasonic diagnostic apparatus can be lessened.

After having calculated the second type of tissue attribute value, the tissue attribute value calculating section 108 outputs the first and/or second type(s) of tissue attribute values (i.e., the two-dimensional map image of radial elasticities and/or that of circumferential elasticities) in accordance with the instruction given by the operator through the user interface 122.

The image synthesizing section 106 synthesizes together the tomographic image provided by the tomographic image processing section 104 and the two-dimensional map image of the radial or circumferential elasticities supplied from the tissue attribute value calculating section 108 and outputs the synthesized image to the monitor 107. In this case, the tomographic image and the two-dimensional map image of elasticities are preferably synthesized together such that two corresponding tissues at the same location are superposed one upon the other.

The memory 121 stores the received signal and at least one of the location tracking information and the magnitude of strain. The memory 121 also stores the radial and circumferential elasticities, which have been calculated by the tissue attribute value calculating section 108, in association with the measuring times. In addition, the memory 121 further stores the tomographic image. With the transmission and reception of the ultrasonic waves suspended, the information stored in the memory 121 is read and used by the tissue attribute value calculating section 108 to calculate the circumferential elasticity. In presenting the circumferential and radial elasticities on the monitor 107, the associated elasticity image data are read from the memory 120 and then synthesized with the two-dimensional map image of the elasticities at the image synthesizing section.

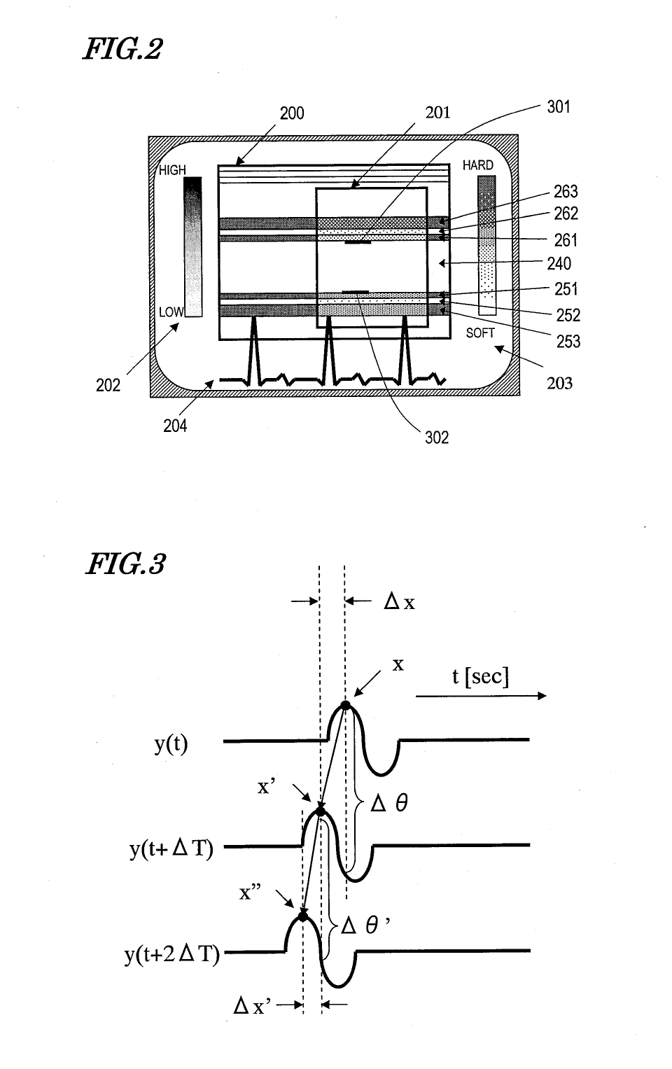

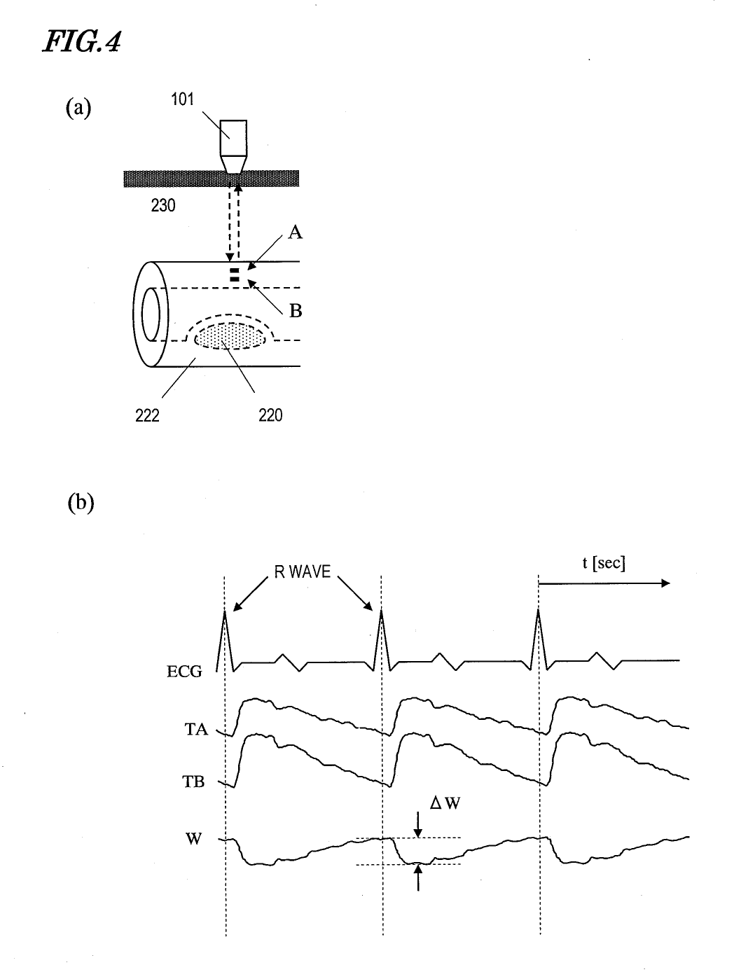

FIG. 2 schematically illustrates a picture that may be presented on the monitor to show exemplary vascular wall elasticities that were measured by an ultrasonic diagnostic apparatus with such a configuration. In FIG. 2, an elasticity image 201, representing the distribution of elasticities at an associated tissue in colors, is superimposed on a vascular wall tomographic image 200 on the monitor. As shown in FIG. 2, an electrocardiographic complex obtained from the subject may be input to the ultrasonic diagnostic apparatus and presented as a biomedical signal waveform 204. Also, as will be described later, cursors 301 and 302 representing the boundary between the vascular wall and the vascular flow are also presented.

The tomographic image 200 is represented by a combination of the reflection intensity indicated by the reflection intensity scale 202 and the gray scale of the image. On the other hand, the elasticity image 201 is represented by a combination of the elasticity indicated by the elasticity scale 203 and the color tone. In FIG. 2, the intima 261, media 262 and adventitia 263 of the vascular anterior wall, the intima 251, media 252 and adventitia 253 of the vascular posterior wall, and vascular lumen 240 are presented on the tomographic image 200 at mutually different gray scales associated with the reflection intensity scale 202. Also, since these tissues have mutually different elasticities, these tissues are also presented on the elasticity image 201 in respectively different color tones associated with the elasticity scale 203.

In transmitting or receiving the ultrasonic wave (which will be referred to herein as a “live state”), the tomographic image 200 is updated at a rate of several tens of frames per second as in a conventional ultrasonic diagnostic apparatus. Meanwhile, as the elasticity is calculated based on a difference in blood pressure every cardiac cycle, the elasticity image 201 is updated once every cardiac cycle.

On the other hand, while the transmission and reception of ultrasonic waves are suspended (which will be referred to herein as a “freeze state”), the received signal, the location tracking information or the magnitude of strain is read from the memory 120, the elasticity is re-calculated, and a tomographic image is read from the memory 120 and presented synchronously with the elasticity at that time. Optionally, in the freeze state, elasticities and tomographic images of the past may also be read. In that case, as disclosed in Patent Document No. 3, at a measuring time that is stored in association with the elasticity, either a tomographic image that was obtained at the same time as the elasticity to present or a tomographic image in the cardiac cycle that it took to calculate the elasticity is read from the memory 120 and presented.

Hereinafter, it will be described how the ultrasonic diagnostic apparatus operates. First, in the live state, the transmitting section 102 and the receiving section 103 are activated to generate received signals at regular intervals. The tomographic image processing section 104 and the tissue tracking section 105 process the received signals sequentially to generate a tomographic image and location tracking information, respectively. Based on these pieces of location tracking information that have been generated sequentially, the tissue attribute value calculating section 108 generates a two-dimensional map image of radial elasticities. Then, the image synthesizing section 106 synthesizes the tomographic image and the two-dimensional map image of radial elasticities together. And the monitor 107 presents the synthesized image thus obtained. In the live state, the ultrasonic waves are transmitted and received one after another, and the tomographic image is updated every time the received signal is generated. The two-dimensional map image of radial elasticities is updated every cardiac cycle. At least one of the received signals, the location tracking information and the elasticities thus obtained is stored in the memory 121. On the other hand, the tomographic images generated are stored in the memory 120.

On the other hand, to calculate a circumferential elasticity, the radius and wall thickness of a blood vessel should be obtained. However, it is difficult to automatically detect the boundary between the vascular wall and vascular flow in a short time. The apparatus is actually allowed a time of just about 100 μs, for example, to process a single received signal. To locate the boundary within such a short time frame, a high-performance computer would be needed. For that reason, the ultrasonic diagnostic apparatus of the present invention calculates only the radial elasticities in the live state.

In the freeze state in which the transmission and reception of ultrasonic waves are suspended, at least one of the received signals, the location tracking information and the radial elasticities is read from the memory 121 and the tissue attribute value calculating section 108 calculates the circumferential elasticities. Also, a tomographic image that was produced at the same time is output from the memory 120 to the image synthesizing section 106. Meanwhile, the radius and wall thickness calculating section 110 determines, based on either the location tracking information or the received signal, the boundary between the vascular anterior and posterior walls and the surrounding tissue or the boundary between the vascular anterior and posterior walls and the vascular flow by the method described above, thereby calculating the radius and wall thickness of the blood vessel, which are output to the tissue attribute value calculating section 108. Even if some delay is caused just after the apparatus has entered the freeze state, that will not make the operator uncomfortable. That is why it may take some time to determine the boundary.

The tissue attribute value calculating section 108 calculates the circumferential elasticities based on the radius and wall thickness of the blood vessel and the location tracking information or the radial elasticities, and then outputs a two-dimensional map image of the circumferential elasticities to the image synthesizing section 106. In response, the image synthesizing section 106 synthesizes together the elasticity image supplied from the memory 120 and the two-dimensional map image of the circumferential elasticities. And the monitor 107 presents the synthesized image thus obtained.

If the radius and wall thickness of the blood vessel have been automatically detected properly, an image including the two-dimensional map image of the circumferential elasticities is automatically presented on the monitor 107 in the freeze state following the procedure described above. However, unless the radius or wall thickness of the blood vessel has been detected properly (i.e., if the boundary of the vascular wall cannot be located exactly), the operator will correct the boundary location or the radius or wall thickness of the blood vessel through the user interface 122. And based on the corrected boundary location or the corrected radius or wall thickness of the blood vessel, the tissue attribute value calculating section 108 calculates the circumferential elasticities.

Even in the freeze state, the data stored in the memories 120 and 121 are read first, and a synthesized image of the tomographic image and the radial elasticities is presented as shown in FIG. 2. In addition, the boundary location that has been calculated by the radius and wall thickness calculating section 110 is also presented on the monitor 107. In FIG. 2, the radius and wall thickness calculating section has located the boundaries between the vascular anterior and posterior walls and the vascular flow and displays the cursors 301 and 302 at those locations.

Using the user interface 122, the operator corrects the locations of the cursors 301 and 302 on the tomographic image on the monitor 107. The radius and wall thickness calculating section 110 gets the corrected locations from the user interface 122 and calculates the radius and wall thickness of the blood vessel. Alternatively, the operator may directly enter the radius and wall thickness of the blood vessel through the user interface 122.

The tissue attribute value calculating section 108 receives either the radius and wall thickness of the blood vessel that have been calculated based on the boundary locations corrected through the user interface 122 or the radius and wall thickness of the blood vessel that have been directly corrected through the user interface 122. Then, the tissue attribute value calculating section 108 calculates the circumferential elasticities and outputs a two-dimensional map image of the circumferential elasticities to the image synthesizing section 106, which synthesizes together the tomographic image supplied from the memory 120 and the two-dimensional map image of the circumferential elasticities. And the monitor 107 presents their synthesized image. As a result, the images on the monitor 107 are changed from the radial elasticities into the circumferential elasticities.

The circumferential elasticity thus obtained is stored in the memory 121 in association with the time when data to calculate the circumferential elasticity was obtained.

In the freeze state, data may start being read from the memories 120 and 121 at an arbitrary point in time of the stored data. If a point in time, of which the circumferential elasticity has already been calculated once by reading data from the memories 120 and 121, is specified, then the circumferential elasticity stored in the memory 121 may be presented at its associated time without calculating the circumferential elasticity in the procedure described above. In this manner, the circumferential elasticity can be presented immediately without calculating it all over again. Optionally, even if the circumferential elasticity can be presented without being calculated again, either the circumferential elasticity or the radial elasticity may be selected in accordance with the operator's instruction.

As described above, the present invention provides an ultrasonic diagnostic apparatus that can calculate radial and circumferential elasticities accurately even without using any special means for carrying out complicated computations to locate the boundary quickly. Consequently, any vascular disease such as arteriosclerosis can be diagnosed exactly using the ultrasonic diagnostic apparatus of the present invention.

In the preferred embodiments described above, radial and circumferential elasticities are supposed to be calculated as the first and second types of tissue attribute values. However, the ultrasonic diagnostic apparatus of the present invention may also calculate any other tissue attribute values as the first and second types of tissue attribute values.

The first type of tissue attribute value could be either a property value that can be calculated relatively easily (i.e., that does not require too much computations) even while ultrasonic waves are being transmitted or received or a property value that can be calculated automatically based on the received signal. Specifically, the first type of tissue attribute value may be the magnitude of strain or a variation in inside diameter.

On the other hand, the second type of tissue attribute value should be either a property value that requires complicated computations or a property value that needs to be calculated in accordance with the corrections made by the operator. Specifically, the second type of tissue attribute value could be a coefficient of viscosity. The coefficient of viscosity μ may be calculated by the following Equation (7) that is disclosed in Non-Patent Document No. 2, for example.

$$μ = \frac{P}{\frac{d ɛ}{d t}} - E ɛ \qquad ( 7 )$$

It is to be noted that P is the pulse pressure of the blood vessel, ε is the strain and E is the elasticity.

On the other hand, the second type of tissue attribute value may be an elasticity E calculated by the following Equation (8) disclosed in Non-Patent Document No. 3, for example.

$$E = \frac{- 3 {Pr}_{i}^{2} {r}_{o}^{2}}{ɛ \cdot 2 ( {r}_{o}^{2} {r}_{i}^{2} ) {r}^{2}} \qquad ( 8 )$$

It is to be noted that P is the pulse pressure of the blood vessel, rᵢ is the inside diameter of the blood vessel, rₒ is the outside diameter of the blood vessel, and r is the distance from the center of the blood vessel. The distance r may be determined by allowing the operator to specify a location where the elasticity needs to be calculated using the user interface 122, for example.

A second type of tissue attribute value like this would require complicated computations. That is why to calculate such a value in the live state (or in real time) with ultrasonic waves transmitted or received, a high-performance arithmetic logic unit or the operator's own input would be needed. That is to say, it is not appropriate to measure such a value in the live state. According to the present invention, however, the second type of tissue attribute value is obtained while the ultrasonic diagnostic apparatus is making no measurements. That is why the computing ability of the ultrasonic diagnostic apparatus that would be used to control the measurements to be done by the apparatus can be used to calculate the second type of tissue attribute value. Also, as there is no need to calculate the second type of tissue attribute value in real time, it may take a while to calculate it. Even so, that would not make the operator feel quite uncomfortable.

Consequently, this ultrasonic diagnostic apparatus can calculate such a tissue attribute value that requires complicated computations even without using a high-performance arithmetic logic unit. Also, if the second type of tissue attribute value is calculated in accordance with the operator's input, he or she can take his or her time and determine and enter the best numerical value while monitoring the tomographic image, for example.

### INDUSTRIAL APPLICABILITY

The present invention contributes effectively to providing an ultrasonic diagnostic apparatus that can calculate the elasticity of a vascular wall accurately.

1. An ultrasonic diagnostic apparatus comprising:
a transmitting section that drives an ultrasonic probe to transmit an ultrasonic wave toward a subject including a blood vessel;
a receiving section that receives an ultrasonic echo, produced by getting the ultrasonic wave reflected by the subject, at the ultrasonic probe to generate a received signal;
a tissue tracking section for tracking the motion of each tissue of the subject based on the received signal and outputting location tracking information;
a radius and wall thickness calculating section that calculates the radius and wall thickness of the blood vessel;
a tissue attribute value calculating section that calculates first and second types of tissue attribute values of the vascular wall of the blood vessel based on at least one of information about the blood pressure of the subject that has been provided externally, the location tracking information, and the radius and wall thickness of the blood vessel; and
a display section that presents the first and second types of tissue attribute values,
wherein the first and second types of tissue attribute values are mutually different types, and
wherein while the transmitting section and the receiving section are transmitting or receiving the ultrasonic wave, the tissue attribute value calculating section calculates the first type of tissue attribute values one after another and the display section presents the first type of tissue attribute values, but
while the transmission and reception of the ultrasonic waves are suspended, the tissue attribute value calculating section calculates the second type of tissue attribute value, and
wherein in accordance with an instruction given by an operator, the tissue attribute value calculating section outputs the first and/or second type(s) of tissue attribute values, and the display section presents the first and/or second type(s) of tissue attribute values supplied from the tissue attribute value calculating section.

2. The ultrasonic diagnostic apparatus of claim 1, wherein the second type of tissue attribute values needs more computations than the first type of tissue attribute values.

3. The ultrasonic diagnostic apparatus of claim 2, wherein the first and second types of tissue attribute values are radial elasticity and circumferential elasticity, respectively, and
wherein while the transmitting section and the receiving section are transmitting or receiving the ultrasonic waves, the tissue attribute value calculating section calculates the radial elasticities one after another based on the location tracking information and the information about the blood pressure, but
while the transmission and reception of the ultrasonic waves are suspended, the tissue attribute value calculating section calculates the circumferential elasticities based on the location tracking information, the information about the blood pressure, and the radius and wall thickness of the blood vessel.

4. The ultrasonic diagnostic apparatus of claim 3, wherein the radius and wall thickness calculating section locates a boundary between the vascular wall and vascular flow and a boundary between the vascular wall and a surrounding tissue based on at least one of the received signal and the location tracking information and calculates the radius and wall thickness of the blood vessel with respect to the boundary located.

5. The ultrasonic diagnostic apparatus of claim 4, further comprising:
a tomographic image processing section that generates, based on the received signal, a tomographic image of the subject to present on the display section; and
a user interface for entering information about the vascular wall-flow boundary and the vascular wall-surrounding tissue boundary into the radius and wall thickness calculating section by getting one of these boundaries specified by the operator on the tomographic image on the display section.

6. The ultrasonic diagnostic apparatus of claim 4, further comprising:
a tomographic image processing section that generates, based on the received signal, a tomographic image of the subject to present on the display section; and
a user interface that allows the operator to enter either corrected ones of the vascular wall-flow boundary and vascular wall-surrounding tissue boundary that have been located by the radius and wall thickness calculating section by getting one of these two boundaries specified by the operator on the tomographic image on the display section or corrected ones of the radius and wall thickness of the blood vessel that have been calculated by the radius and wall thickness calculating section.

7. The ultrasonic diagnostic apparatus of claim 5, wherein while the ultrasonic waves are not being transmitted or received, the display section continues to present the first type of tissue attribute value until the operator enters information into the radius and wall thickness calculating section through the user interface.

8. The ultrasonic diagnostic apparatus of claim 7, further comprising a memory that stores at least one of the received signal, the location tracking information and the magnitude of strain calculated based on the location tracking information,
wherein while the ultrasonic waves are not being transmitted or received, the tissue tracking section and the tissue attribute value calculating section read at least one of the received signal, the location tracking information and the magnitude of strain from the memory, calculate the second type of tissue attribute value, and present the value on the display section.

9. The ultrasonic diagnostic apparatus of claim 8, wherein the tissue tracking section and the tissue attribute value calculating section read at least one of the received signal, the location tracking information and the magnitude of strain calculated based on the location tracking information at an arbitrary point in time from the memory, calculate the second type of tissue attribute value, and present the value on the display section.

10. The ultrasonic diagnostic apparatus of claim 9, wherein the memory stores the second type of tissue attribute value calculated in association with the time.

11. The ultrasonic diagnostic apparatus of claim 10, wherein if the second type of tissue attribute value is stored in the memory, the tissue attribute value calculating section reads the second type of tissue attribute value from the memory without receiving the operator's input through the user interface and the display section presents the second type of tissue attribute value.
